(19) United States
(12) Patent Application Publication
(10) Pub. No.: US 2018/0256560 A1
(43) Pub. Date: Sep. 13, 2018

(54) LIQUID NICOTINE FORMULATION

(71) Applicant: TTP plc, Melbourn Hertfordshire (GB)

(72) Inventors: Hans-Jürgen Hoppe, Melbourn Hertfordshire (GB); Iain McDerment, Melbourn Hertfordshire (GB)

(21) Appl. No.: 15/761,931

(22) PCT Filed: Sep. 22, 2016
(86) PCT No.: PCT/GB2016/052958

§ 371 Date: Mar. 21, 2018

(30) Foreign Application Priority Data
Sep. 22, 2015 (GB) 1516729.9

(51) Int. Cl. A61K 31/465; A61K 9/00; A61K 47/10; A61K 9/12

(52) U.S. Cl. CPC A61K 31/465; A61K 9/0078; A61M 15/009; A61K 9/12; A61K 47/10

(57) ABSTRACT

There is disclosed a liquid formulation for inhalation into the lungs, comprising an aqueous solution of nicotine or a nicotine analogous molecule, at least one organic and/or inorganic salt, an organic liquid having a viscosity higher than water, and optionally an organic alcohol, wherein the pH of the formulation is greater than pH 7 and the formulation does not include a propellant. There is further disclosed a corresponding method of aerosolising the liquid formulation, a method for delivering nicotine to the user, and an aerosol-generating device.

### FIELD OF THE INVENTION

The present invention relates to a liquid nicotine formulation for delivering nicotine to a subject by inhalation, such as a nicotine delivery formulation administered to the lungs as an aerosol. In addition, the present invention is directed to an aerosol-generating device comprising the liquid nicotine formulation and a method of delivering nicotine to a user. In particular, the invention relates to formulations of nicotine composed to achieve cigarette-like nicotine delivery effects, specifically in terms of rapid onset (sub 5 min nicotine peak delivery to the brain) and delivered amount (greater than 10 ng/ml peak nicotine in arterial blood).

### BACKGROUND

To reduce the adverse effects on health resulting from tobacco smoking, nicotine delivery products are becoming an increasingly popular alternative to cigarette use and are being advocated by governments and non-governmental organisations.

Current versions of such devices (electronic or e-cigarettes and other vaping devices) produce aerosols with large amounts of nicotine, but in formulations and aerosol types poorly suited to efficient nicotine delivery. For example, Benowitz et al., (1988), Clin. Pharmacol. Ther. 44:23-28 compares the pharmacokinetics elicited by smokeless nicotine consumption with that of real cigarettes and notes the discrepancy in delivery site, as well as in pharmacokinetic performance. A dominant reason for poor performance of current smokeless nicotine delivery technologies lies in the site and quantity of nicotine absorption in the airways.

Other formulations of nicotine for different replacement products also exist, but show even poorer pharmacokinetic performance than e-cigarettes (e.g. patches, nose sprays, mouth sprays, gums).

In general, no consumer nicotine delivery mechanism exists with cigarette-like nicotine delivery kinetics. However, Shao et al., Nicotine & Tobacco Research, 2013; 1248-1258 demonstrates from laboratory studies in rats demonstrated cigarette-like nicotine kinetics when measured in arterial blood when nicotine was administered using a high-power nebulizer capable of producing sub μm droplets containing nicotine in gas-capable form, demonstrating the feasibility in principle, to design a formulation for human consumption capable of producing cigarette-like nicotine delivery kinetics.

Given the poor performance of current nicotine replacement inhalers, understandable consumer demands for ever higher nicotine concentrations and formulation doses are being regulated to limit an upper nicotine concentration of 20 mg/ml and a maximal dose of 2 ml per cartridge (DIRECTIVE 2014/40/EU OF THE EUROPEAN PARLIAMENT AND OF THE COUNCIL of 3 Apr. 2014 and repealing Directive 2001/37/EC).

Up to now, what is not known is a nicotine formulation within this regulated range which is capable of mimicking the nicotine pharmacokinetics of actual cigarette smoking by using an inhalation device, i.e. without smoking a cigarette.

The principal problem of nicotine delivery through aerosols lies in the delivery to the alveoli, i.e. the specialized area of the lung with the largest gas exchange surface, of a sufficient amount of nicotine in its uncharged from, i.e. the form capable of release from droplets as gaseous nicotine (Pankow, Chem. Res. Toxicol. 2001; 14(11):1465-81).

Theoretical and experimental studies indicate that droplets and particles reach respiratory sections of the lung according to their diameter: >10 μm droplets target mouth and throat, 7-5 μm droplets target the trachea bronchi and upper bronchioli, 3 μm droplets target respiratory bronchioli, and 1 μm and smaller droplets target alveoli. However, sub-μm droplets do not deposit effectively into the lung surfactant and are often exhaled (Morawska et al. J Aerosol Sci. 2008; 40:256-269). Excipient enhanced growth of droplets through the adsorption of water from the surroundings, highly humid air in the lower respiratory tract is known to allow growth of droplets beyond exhalable sizes and is mediated by hygroscopic concentrations of salts in the formulation (Longest P W et al. Aerosol Sci. Technol. 2011 Jan. 1; 45(7):884-899). The generation of 1 μm and smaller droplets, however, is still required, but also demands significantly larger amounts of energy, increasing the difficulties to develop hand-held consumer products with reasonable user experiences to target this droplet size.

First physiological effects of nicotine delivery from cigarette smoke arise very rapidly (about 15 seconds), a reaction time which is believed to be too short for diffusion governed transport of dissolved, charged nicotine molecules. Instead, the gaseous form of uncharged nicotine is thought to contribute most to the rapid pharmacokinetic effect of nicotine inhalation. However, formulations for enhanced release of gaseous nicotine from solutions are currently not known.

### SUMMARY OF THE INVENTION

The present invention provides a liquid formulation for inhalation into the lungs, comprising an aqueous solution of nicotine or a nicotine analogous molecule capable of binding to a Nicotinic Acetylcholine Receptor, at least one organic and/or inorganic salt, an organic liquid having a viscosity higher than water, and less than 50% vol/vol (based on the volume of the liquid formulation) of an organic alcohol,

wherein the pH of the liquid formulation is greater than pH 7 and the formulation does not include a propellant.

The pH of the liquid formulation may be between pH 7.5 and 14, such as between 7.5 and 9.5.

The liquid formulation may comprise at least one organic and/or inorganic salt selected from the group consisting of sodium chloride, metal citrates, metal sulphates and combinations thereof. The at least one inorganic salt may be sodium chloride.

The organic liquid may be selected from the group consisting of glycerol, glycol, a carbohydrate solution and combinations thereof. The organic liquid may be glycerol or glycol (e.g. ethylene glycol or polyethylene glycol). The liquid formulation may include the organic liquid in an amount of from 0.5 to 10% vol/vol, such as 0.5 to 5% vol/vol, or 0.5 to 2% vol/vol.

The concentration of the organic or inorganic salts may be between 0.05 and 2 molar, such as between 0.1 and 0.3 molar.

The organic alcohol may be ethanol. The liquid formulation may comprise ethanol in an amount from 0.5 to 20%, such as 0%-5%, 0.5-10% or 2-10% vol/vol.

The liquid formulation may comprise: nicotine in an amount of between 2-20 mg/ml; at least one organic or inorganic salt, such as NaCl, having a concentration of 0.05-2M; 0.5-2% vol/vol of at least one organic liquid, such as glycerol or glycol; 1-10% vol/vol of ethanol, and wherein the liquid formulation has a pH greater than pH 7.

The liquid formulation may further comprise an additive, such as a flavouring, a colourant, an antioxidant, or a surfactant.

The liquid formulation may be dispensed with an aerosol-generating device to form an aerosol. The aerosol may contain droplets having a mean diameter of 10 μm or less and/or a surface to volume ratio of 0.6 μm⁻¹ or greater, preferably a mean diameter of 4 μm or less and/or a surface to volume ratio of 1.5 μm⁻¹ or greater. The liquid formulation may comprise between 1.5 and 8 μg/μl of nicotine.

In a second aspect of the present invention, there is provided an aerosol-generating device comprising a reservoir which contains the liquid formulation of the present invention. The device may comprise an atomiser or a nebuliser. The device may be configured to generate an average droplet size of 10 μm or less (i.e. 10 μm to 200 nm), preferably 5 μm or less (i.e. 5 μm to 200 nm), 3 μm or less (i.e. 3 μm to 200 nm), 2 μm or less (i.e. 2 μm to 200 nm) or 1 μm or less (i.e. 1 μm to 200 nm), when activated to aerosolise the liquid formulation. The device may be configured to generate a droplet size having an average surface to volume ratio of between 0.6 μm⁻¹ and 30 μm⁻¹, preferably 1.25 μm⁻¹ and 15 μm⁻¹, when activated to aerosolise the liquid formulation.

In another aspect, the present invention is directed to a method for delivering nicotine to a user by inhalation, the method comprising the steps of:

(a) administering an aerosol of the liquid formulation of the present invention to the user, and
(b) allowing the nicotine to be delivered to the arterial blood.

### BRIEF DESCRIPTION OF THE FIGURES

The invention will now be described in more detail by way of example only, and with reference to the following figures.

FIG. 1.1 illustrates the chemical structure of nicotine and its pH dependent forms.

FIG. 1.2 is a series of plots of relative nicotine % versus pH showing the relative concentration of each nicotine form at a given pH.

FIG. 1.3 is a series of plots of absorbance versus concentration showing nicotine UV absorption at low pH in ethanol shows good correlation with concentration.

FIG. 2.1 is a schematic representation illustrating a method for determining the amount of nicotine vapour at equilibrium.

FIG. 2.2 is a graph illustrating the effect of pH on equilibrium gaseous nicotine.

FIG. 2.3 is a graph illustrating the effect of ethanol on equilibrium gaseous nicotine concentration.

FIG. 3.1 is a schematic representation illustrating a method for determining nicotine vapour under dynamic evaporation conditions.

FIG. 3.2 is a graph illustrating the effect of pH on dynamic gaseous nicotine release.

FIG. 3.3 is a graph illustrating the effect of salt (NaCl) on dynamic gaseous nicotine.

FIG. 3.4 is a graph illustrating the effect of ethanol on equilibrium gaseous nicotine.

FIG. 4.1 is a series of plots of average droplet size versus ethanol % showing the effect of ethanol on size of droplet generation.

FIG. 4.2 is a series of plots of average droplet size versus glycerol % showing the effect of glycerol on size of droplet generation.

FIG. 4.3 is a series of plots of average droplet size versus NaCl concentration showing the effect of salt (NaCl) on size of droplet generation.

FIG. 4.4 is a series of plots of average droplet diameter versus glycerol % showing the effect of salt on size of droplet generation in the presence of 10% ethanol.

FIG. 4.5 is a series of plots of average droplet diameter versus glycerol % showing the effect of glycerol on size of droplet generation in the presence of 1% ethanol.

FIG. 4.6 is a series of plots of average droplet diameter versus salt [M] showing the effect of salt on size of droplet generation in the presence of 1% and 10% of ethanol.

FIG. 4.7 is a series of plots of average droplet diameter versus pH showing the effect of pH on size of droplet generation.

FIG. 5 depicts a conventional nebuliser/atomiser which can be used to aerosolise the liquid formulation of the present invention. FIG. 5 is depicted in U.S. Pat. No. 3,812,854 (the contents of which are incorporated herein) as FIG. 1. The description of FIG. 1 in U.S. Pat. No. 3,812,854 in column 3 is referred to.

FIG. 6 depicts the results of an experiment comparing the nicotine remaining in droplets.

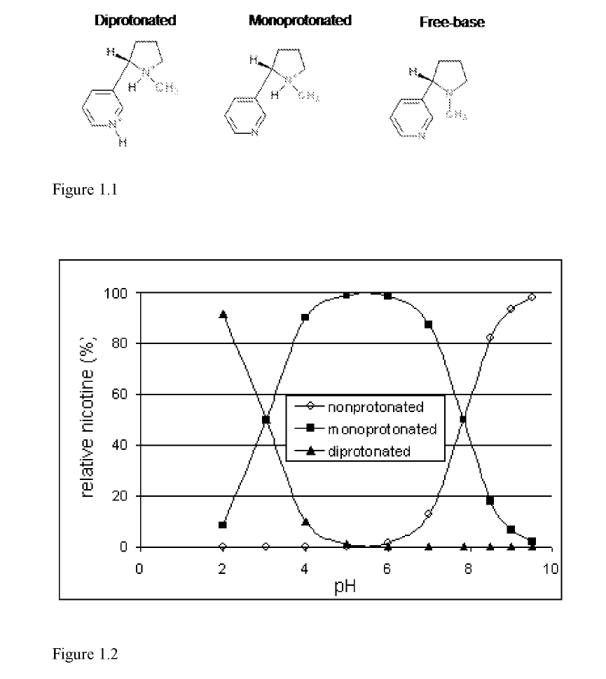

### DESCRIPTION OF EMBODIMENTS

In contrast to the foregoing difficulties in delivering nicotine with cigarette-like efficiency through traditional inhalers, vaporisers and their corresponding formulations, the present invention provides a formulation which allows a known, low energy consuming, hand-held aerosol generating inhaler to deliver sufficiently small droplets to reach the alveoli in a form that allows for both, enhanced nicotine gas release and excipient enhanced growth of droplets for alveolar retention; the latter also ensuring the absence, or significant minimization, of nicotine in the exhaled air.

To achieve these effects, nicotine may be incorporated into the formulation without pH adjustment so that it exists predominantly in uncharged form, equivalent to around pH 10 (>99% uncharged). This allows for the largest possible fraction of delivered nicotine to participate in gas exchange.

Any suitable source of nicotine may be employed. For example, the nicotine may be a nicotine free base or a nicotine derivative. The nicotine derivative may be any nicotine analogous molecule which is capable of binding to Nicotinic Acetylcholine Receptors. Suitable nicotine analogous molecules include acetylcholine, choline, epibatidine, iobeline, varenicline and cytisine. The liquid formulation may comprise nicotine in an amount of between 0-20 mg/ml, 2-20 mg/ml, 2-15 mg/ml, 1-8 mg/ml or 1.5-6 mg/ml.

The liquid formulation comprises an organic alcohol (e.g. ethanol), in an amount less than 50% by weight of the liquid formulation. For example, the liquid formulation may not comprise an organic alcohol at all or an organic alcohol other than ethanol may be present. If the organic alcohol (e.g. ethanol) is present in the formulation, it may be present in an amount between 0.5% and 35%, such as 1% to 25%, 1% to 10%, or in an amount less than 10% vol/vol (based on the volume of the liquid formulation). In an embodiment of the invention, the liquid formulation does not comprise ethanol.

In the context of the present invention, the term “organic alcohol” includes primary, secondary and tertiary alcohols as well as polyols, such as diols and glycols. Suitable organic alcohols which can be used in the present invention include ethanol and diols. The organic alcohol may be a C₁-C₁₆ alcohol, preferably a C₁ to C₆ alcohol, such as ethanol. The alcohols may be linear or branched. Ethanol is referenced throughout the specification but the skilled person would appreciate that alternative organic alcohols, such as those mentioned above, may be used instead.

It has been found that ethanol evaporates from ethanol/water and ethanol/water/glycerol mixtures very rapidly and evaporates fastest at lower concentrations. In addition, it has been found that as ethanol reduces the viscosity and surface tension of aqueous solutions, which in turn negatively affects small droplet generation, an experimentally determined amount (which is within the abilities of a person skilled in the art) of viscosity and surface tension enhancing substances may also be added to the formulation, such as 0.5%-10%, 0.5%-5%, 0.5%-2% or 1-2% vol/vol (final) glycerol or glycol, or 0.5%-10%, 0.5%-5%, 0.5%-2% or 1-2% wt/wt sorbitol.

The liquid formulation comprises at least one organic liquid, wherein the organic liquid has a viscosity higher than water. (The dynamic viscosity of water is 8.90×10⁻⁴ Pa·s at about 25° C.) Suitable organic liquids include compounds which when mixed with water increase the viscosity of the mixture according to Refutas' equation. For example, the at least one organic liquid may be ethylene glycol (1.61×10⁻² Pa·s), glycerol (1.2 Pa·s), a carbohydrate solution or combinations thereof. Suitable carbohydrate solutions include glucose, sorbitol and saccharose. The organic liquid may be ethylene glycol, polyethylene glycol or glycerol. The organic liquid may be included in the formulation in the range of 0.5 to 10% vol/vol, such as 0.5 to 6%, or 0.5 to 2% vol/vol (based on the volume of the liquid formulation). The ratio of ethanol to glycol or glycerol in the liquid formulation of the present invention may be in the range of 10:1 to 25:1.

The liquid formulation comprises at least one organic/inorganic salt. Suitable salts include sodium chloride (NaCl), metal phosphates, metal tartrates, metal malates, metal lactates, metal citrates, or metal sulphates. For example, the inorganic salt may be sodium chloride. It has been found experimentally that increasing the concentration of the salt enhances the gaseous release of nicotine from the solution (1M NaCl: two-fold). Biocompatible salts, such as sodium chloride, citrate, or sulphate enhance nicotine gas release significantly at concentrations at which they also enhance excipient-mediated growth of droplets. Therefore, the formulation may contain such salts within the range of 0.05-2M. The combined concentration of the organic or inorganic salts may be between 0.05 and 2 molar, or 0.1 and 0.3 molar.

The liquid formulation of the present invention does not comprise a propellant. In the context of the present invention, the term “propellant” refers to a compound, such as a HFA (hydrofluoroalkane) propellant, typically having a boiling point in the region of minus 100 to +30 degrees centigrade and a density of 1.2 to 1.5 g/cm³, a vapour pressure of 40-80 psig and which are non-flammable and non-toxic to human inhalation. A propellant in the context of the present invention is a chemical substance used in the production of pressurised gas that is subsequently used to create movement of a fluid when the pressure is released.

In an embodiment of the invention, the liquid formulation does not include an additional buffering agent (i.e. although nicotine may be considered to be a buffering agent, the formulation does not include a further buffering agent). In the context of the present invention, the term “buffering agent” refers to a weak acid or base used to maintain the acidity of a solution near a chosen value after the addition of another acid or base.

The pH of the liquid formulation may be between pH 7.5 and 14. For example, the pH of the liquid formulation may be between pH 7.5 and 9.5.

The inventors have found that nicotine delivery may be controlled by adjusting: (i) the pH of the liquid formulation; (ii) the amount of nicotine contained in the liquid formulation; and (iii) the size of the droplets produced when the liquid formulation of the present invention is atomised. For example, the inventors have found that a large amount of nicotine moves into the gas phase at high pH, such as between pH 8 and 14. The availability of gaseous nicotine can be reduced by reducing the pH of the liquid formulation. As such, the present invention allows for tailored gaseous nicotine release.

In an embodiment of the invention, the pH of the liquid formulation is between 7.5 and 8.5 and the liquid formulation contains nicotine in an amount of between 8 μg/l and 20 μg/μl. In another embodiment, the pH of the liquid formulation is between 8.5 and 14 and the liquid formulation contains nicotine in an amount of between 0 μg/l and 8 μg/μl, preferably between 1 μg/l and 6 μg/μl.

Nicotine is known to exert its effect on neural cells in the brain and a rapid increase in arterial nicotine concentration is known to characterise cigarette-like pharmacokinetics of nicotine and mediate its effects. For this rapid rise in arterial blood nicotine concentration to occur, nicotine needs to pass rapidly from inhaled air in the lungs into the bloodstream. While charged nicotine molecules (the majority of nicotine at pH <8) are not volatile and diffuse slowly through lung surfactant as a dissolved salt, uncharged nicotine can move into the gas phase and rapidly diffuse through membranes and into the blood stream. The amount of uncharged nicotine in a given solution is dependent on the combination of the pH of the formulation and the nicotine concentration. For example, at pH 7, approximately 10% of nicotine molecules are uncharged, whereas at pH 10, almost 100% are uncharged. A solution containing 20 mg/ml nicotine at pH 7 thus contains an equal amount of uncharged nicotine to a 2 mg/ml solution at pH 10.

The liquid formulation of the present invention may comprise: nicotine in an amount of between 2-20 μg/μl; at least one organic or inorganic salt, such as NaCl, having a concentration of 0.05-2M; 0.5-2% vol/vol of at least one organic liquid, such as glycerol or glycol; and 1-10% vol/vol of ethanol, wherein the pH of the liquid formulation is between pH 7 and 14.

The formulation may further comprise one or more additives. For example, the liquid formulation of the present invention may comprise a flavour component. Suitable flavour components include those flavour components typically added to tobacco products. For example, the flavour component may be menthol, fruity, coffee, tobacco or sweet. Typically, the concentration of the flavouring component is chosen such that it will not affect either nicotine gas release or droplet size. Alternatively, or additionally, the liquid formulation of the present invention may comprise a throat impact enhancing substance, such as citric acid. In the context of the present invention, the term “throat impact enhancing substance” refers to a substance that modulates the throat impact feel of the formulation (e.g. “harshness” or “catch”). Alternatively, or additionally, the liquid formulation may comprise a colourant, such as caramel. Alternatively, or additionally, the liquid formulation may comprise a sweetener, such as glucose. Alternatively, or additionally, the liquid formulation may comprise an antioxidant, such as vitamin E. Alternatively, or additionally, the liquid formulation may comprise a surfactant, such as a phospholipid (e.g. oleic acid, lecithin, Span 85, PVP K25). Additionally, or alternatively, the liquid formulation may comprise a pH adjuster, such as HCl, which will dissociate in solution.

The nicotine contained in the liquid formulation may be predominantly uncharged (for example, less than 15%, 10% or 5% of the nicotine contained in the formulation may be charged).

The inventors have found that when the formulation of the present invention is inhaled orally, it is able to better mimic the gaseous nicotine release required for a pharmacokinetic profile of nicotine generated by smoking of a conventional cigarette, when compared to previously known nicotine compositions/formulations.

The liquid formulation can be used in conjunction with an aerosol-generating device which converts the liquid formulation into an aerosol/vapour which can be inhaled by the user via a mouthpiece. Suitable aerosol-generating devices which can be used in the present invention include jet nebulisers, electronic nebulisers (such as those disclosed in U.S. Pat. No. 3,812,854 and U.S. Pat. No. 5,518,179) and mechanical aerosolisation devices. The mechanical aerosolisation devices may generate small droplets via the natural break-up of a jet into droplets (Rayleigh break-up), via the impingement of two jets, such as those disclosed in U.S. Pat. No. 5,472,143 and PCT/GB2015/053221, via the impingement of a fluid stream onto a surface, as disclosed in PCT/GB2015/051413 or via the introduction of instability, with a swirl chamber, in a pressure swirl spray nozzle.

In the context of the present invention, the term “aerosol” refers to a colloid of liquid droplets in air or another gas to be dispensed in a cloud or mist.

When the liquid formulation of the present invention is aerosolised, the aerosol may contain droplets having a mean diameter of between 10 μm and 0.4 μm and/or a surface to volume ratio between 0.6 μm⁻¹ and 15 μm⁻¹, preferably between 4 μm and 0.5 μm and/or a surface to volume ratio between 1.5 μm⁻¹ and 9 μm⁻¹, optionally wherein the formulation comprises between 1.5 and 8 μg/μl of nicotine.

The aerosol-generating device used to aerosolise the liquid formulation of the present invention may comprise a reservoir which contains the liquid formulation. The device may be configured to generate an average droplet size between 10 μm and 200 nm, preferably between 5 μm and 400 nm, when activated to aerosolise the liquid formulation. The device may be configured to generate a droplet size having an average surface to volume ratio of between 0.6 μm⁻¹ and 30 μm⁻¹, preferably 1.25 μm⁻¹ and 15 μm⁻¹, when activated to aerosolise the liquid formulation.

The inventors have found that a nicotine formulation in the form of an aerosol which comprises droplets which have an average surface to volume ratio of greater than 0.6 μm⁻¹, preferably greater than 1.5 μm⁻¹, can deliver to the lungs more than 25 μg of uncharged nicotine per minute. Furthermore, a nicotine formulation containing between 1.5 and 8 μg/μl predominantly uncharged nicotine in the form of an aerosol which delivers to the lungs more than 80 μg uncharged nicotine over any 3 minute period in a liquid form which exhibit a surface to volume ratio of greater than 0.6 μm⁻¹, preferably greater than 1.5 μm⁻¹.

In the context of the present invention, the term “diameter” encompasses the largest dimension of a droplet. The terms “average droplet size”, “mean diameter” and “average diameter” refer to volume median diameter, and specifically the DV 0.5 (or DV 50) value (which is a standard value which can be obtained using, for example, a Malvern Spraytec apparatus). The Volume Median Diameter (VIVID) refers to the midpoint droplet size (mean) (i.e. DV 0.5), where half of the volume spray is in droplets smaller, and half of the volume is in droplets larger than the mean. A VIVID (DV 0.5) of 400, for example, indicates that half of the volume is in droplet sizes smaller than 400 microns, and half the volume is in droplet sizes larger than 400 microns.

The formulation of the present invention may allow the generation of small droplets using a device generating a liquid stream which is allowed to impact a baffle or another such stream.

The formulation of the present invention also allows for droplet size reduction through evaporation of liquid after droplet generation, specifically of ethanol and water, so that the majority of droplets pass through the throat area and into the lung. Furthermore, it allows droplets to increase in size through the acquisition of water from the humidity contained in the airways of the lung, predominantly brought about by the formulation comprising a dissolved salt at concentrations greater than that found in physiological fluids, such as lung surfactant or serum.

### Materials

Nicotine ((−)-Nicotine, ≥99% (GC), liquid, Synonym: (−)-1-Methyl-2-(3-pyridyl)pyrrolidine, (S)-3-(1-Methyl-2-pyrrolidinyl)pyridine) was obtained from Sigma-Aldrich (N3876).

The functional formulation additives ethanol (Ethyl alcohol, Pure, 200 proof, ACS reagent, ≥99.5%); glycerol (Glycerol, ≥99.5%); NaOH (Sodium hydroxide solution volumetric, 4 M NaOH (4N) Fluka), NaCl (Sodium chloride, puriss. p.a., ≥99.5% (AT)); and as solution (Sodium chloride solution, 5M in H₂O); and HCl (Hydrochloric acid concentrate for 10 L standard solution, 1 M HCl (1N)) were all obtained from Sigma-Aldrich ((459844), (G9012), (71535), (71380), (S5150) and (38283), respectively).

### EXPERIMENTAL METHODS

### Example 1—Synthesis of Liquid Nicotine Formulation

(−) Nicotine liquid was diluted in ethanol and/or de-ionized water to yield a stock concentration of 40 mg/ml, before being stored at 4° C. until use. Absolute ethanol, sodium chloride (5M solution), glycerol and de-ionized water were then mixed with the nicotine stock solution to produce the test solutions detailed in the experiments. The pH of the test solution was adjusted to the desired level using 1M HCl.

### Example 2—Static Experiments

3-10 ml of test solution was left to equilibrate with 40-47 ml headspace (air) within a Pyrex glass tube (Pyrex quickfit MF 24/3) sealed with two layers of Parafilm (PARAFILM® M, roll size 4 in.×250 ft, from Sigma-Aldrich (P7668)) overnight (at least 12 hours). To analyse the amount of nicotine in the headspace above each solution at equilibrium, three times three ml of air were sampled through three ml of acidified ethanol (20 mM HCl) in absolute ethanol in a 5 ml glass syringe (Hamilton, model 1005), shaking the mixture in the syringe extensively for 30 seconds after each sample and measuring the absorption of the captured nicotine in the acidified ethanol at 262 nm in a UV/VIS spectrophotometer (Jenway 6715) equipped with a 10 mm quartz cuvette (Hellma Quartz Cell 110-10-40). All experiments except UV measurements were carried out at room temperature (20° C.) in an extractor fume cupboard.

### Example 3—Dynamic Experiments

A constant stream of air at 2.51/min was generated by a Welch pump (model 2546C-02) and sucked over 10-20 ml of test solution at the bottom of a 400 ml Pyrex Erlenmeyer flask which had a PFTE tube placed at 50 mm distance from the centre of the test solution surface. Nicotine released from the test solution was then carried by air stream through a wash bottle with a fritted inlet and nicotine contained in the stream captured by 30 ml of acidified ethanol. Captured nicotine amounts were determined by measuring the UV absorption of the capture solution at 262 nm in a UV/VIS spectrophotometer (Jenway 6715) equipped with a 10 mm quartz cuvette (Hellma Quartz Cell 110-10-40). Nicotine release rates were then calculated by dividing the total amount of nicotine captured by the time of capture. All experiments except UV measurements were carried out at room temperature (20° C.) in an extractor fume cupboard.

### Example 4—Droplet Size

Test solutions were prepared from ethanol, de-ionized water, a 20% stock solution of glycerol in de-ionized water, a 5 M NaCl solution and nicotine solutions of 40 mg/ml in either ethanol or de-ionized water. Solutions were analysed for droplet size distributions generated by an eFlow© rapid nebulizer device (PARI GmbH) which was placed at 90° to, and 15 cm from, the laser light path of a Malvern Spraytec particle sizer. A Malvern Spraytec Real Time Droplet Sizer and an Alberta Idealized Throat coupled to an Andersen Cascade Impactor (AIT/ACI) were also used to determine droplet size.

### Example 5—Surface to Volume Ratios

The surface to volume ratio of the droplets was calculated using the following formulae:

Formula for determining the surface of the droplet: A=4πr²

Formula for determining the volume of the droplet: V=(4/3)πr³

Surface to Volume ratio=A/V

Wherein A stands for surface area, V for volume and r for the radius of the droplet.

Alternative Calculation of Surface/Volume Ratios

A particle sizer like the Malvern Spraytec determine read out the percentage of volume of droplets under a certain DV 0.5 mean diameter. Typically, the flow rate (volume per minute) of a given aerosol generator is also known or easy to measure (spray for a minute, measure liquid missing). So by multiplying % of volume sprayed as droplets under a certain diameter with the flow rate one can easily determine the volume in ml which contains the nicotine capable of contributing to arterial nicotine peak generation. For example, at a flow rate of 1 μl per second, if 80% of droplets are smaller than 5 μm in diameter and have been generated from a formulation containing 2 mg/ml unprotonated nicotine (i.e. at pH 10) then by inhaling the equivalent of 1 minute of that spray one would receive 60 min×1 μl×2 μg/μl×0.8=96 μg of nicotine. Assuming complete uptake of gaseous nicotine, a 3 minute delivery would be enough to produce a ‘hit’ of 50 ng/ml in 5 litres of arterial blood.

Experimental Results

Static Experiments

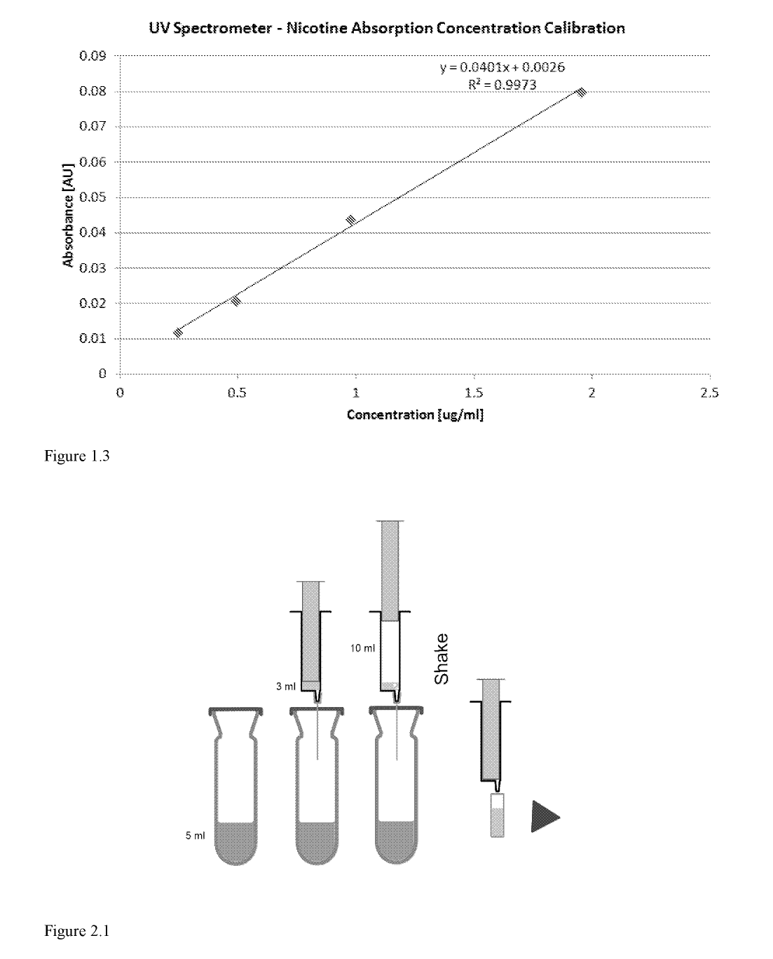

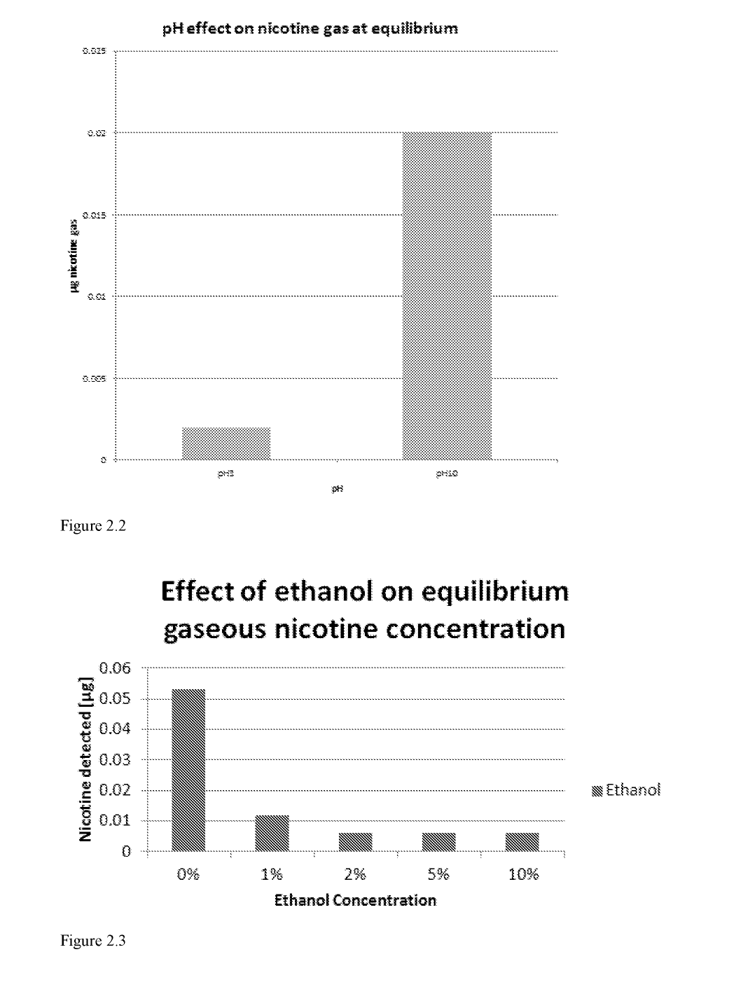

The amount of nicotine released into the gaseous phase from a nicotine solution under equilibrium conditions reflects the vapour pressure of nicotine under these conditions and reflects the overall effect of gas release and re-absorption rates when in equilibrium. As only the formulation is being varied in the experiments, the relative impact on nicotine gas pressure from a solution by its co-solvent/additive can be determined. It was found that the pH of the solution greatly affected nicotine release, with high pH promoting and low pH decreasing gaseous nicotine. For example, FIG. 2.2 shows that decreasing the pH of the liquid formulation from 10 to 3 dramatically decreases the amount of nicotine in gas form. This demonstrates that the relative concentration of un-protonated and hence un-charged nicotine, the form capable of moving into the gas phase, diminishes at lower pH values.

At 20 mg/ml nicotine, it was also found that increasing ethanol concentration very effectively blocks gaseous nicotine, most dramatically at low (below 10%) concentrations. FIG. 2.3 demonstrates that increasing the ethanol concentration from 0 to 100% dramatically decreases the nicotine in gas form, with 90% of reduction being achieved with only 1% ethanol. This observation has not been published previously.

In addition, the impact of the metal salt on the amount of gaseous nicotine is dramatic, with higher salt concentrations resulting in larger amounts of gaseous nicotine.

As the gas capable form of nicotine is uncharged, the salt effect could be explained by the ionic strength of the solution driving gas release through the greater inability of the solvent to effectively hydrate dissolved nicotine molecules. Again, this observation has not previously been reported.

Dynamic Experiments

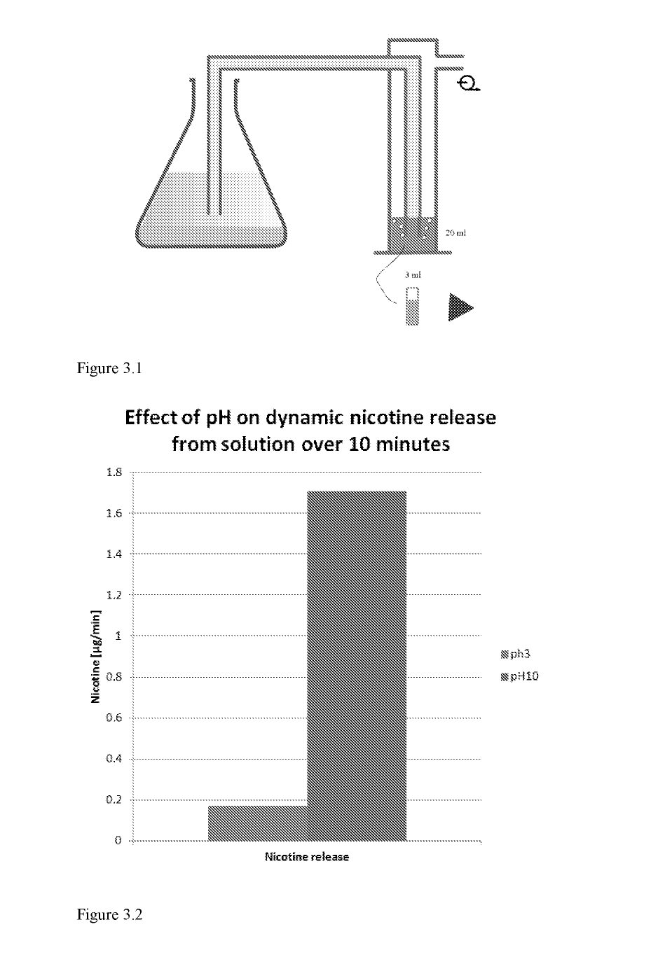

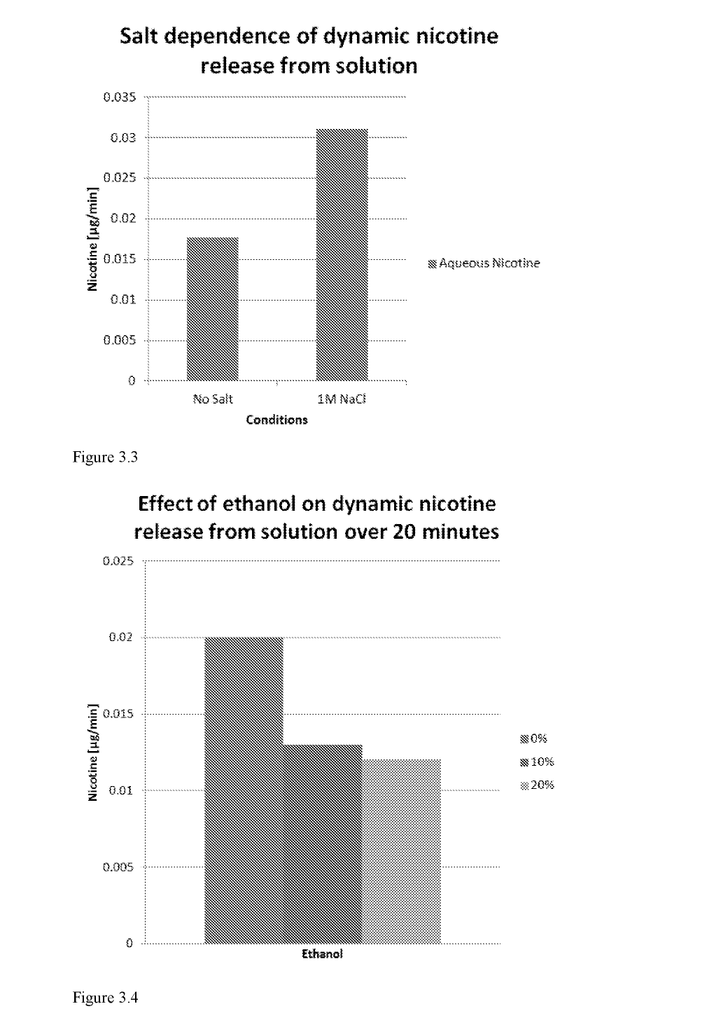

The dynamic data correspond well with the static data obtained at the gas/solution equilibrium. For example, as is evident from FIG. 3.2, decreasing the pH from 8 to 3 dramatically decreases the nicotine gas release. Similarly, FIG. 3.3 demonstrates that increasing the NaCl concentration form 0 to 1M dramatically increases the nicotine release in gas form. In relation to the effect of ethanol on dynamic nicotine release, FIG. 3.4 demonstrates that increasing the ethanol concentration from 0 to 100% dramatically decreases the nicotine gas release, with 90% of reduction being achieved with only 10% ethanol.

However, it was found that the focus on gas release by largely removing the opportunity for nicotine to re-absorb into the solvent. The experimental data hence sheds light on the opportunity of formulation constituents to modulate the rate of nicotine release from solutions.

Droplet Size

Droplet sizes generated (eFlow) can be manipulated effectively through changes in the chemical composition of the droplets. Using a rational approach to address the unmet needs identified (creating small droplets to reach the alveoli, grow on their way towards the alveoli to a size not being exhaled and to do so while enhancing nicotine gas release), optimal concentration ranges for ethanol, glycerol, salt and pH were identified in the experiments detailed. Specifically, an organic liquid (e.g. glycerol) concentration of 0.5 to 2%, an organic alcohol (e.g. ethanol) concentration of 1-10%, a salt concentration of 0.05-2M and a high pH have been found to produce a formulation suitable for both, efficient nicotine release and droplet sizes suitable for targeting alveoli.

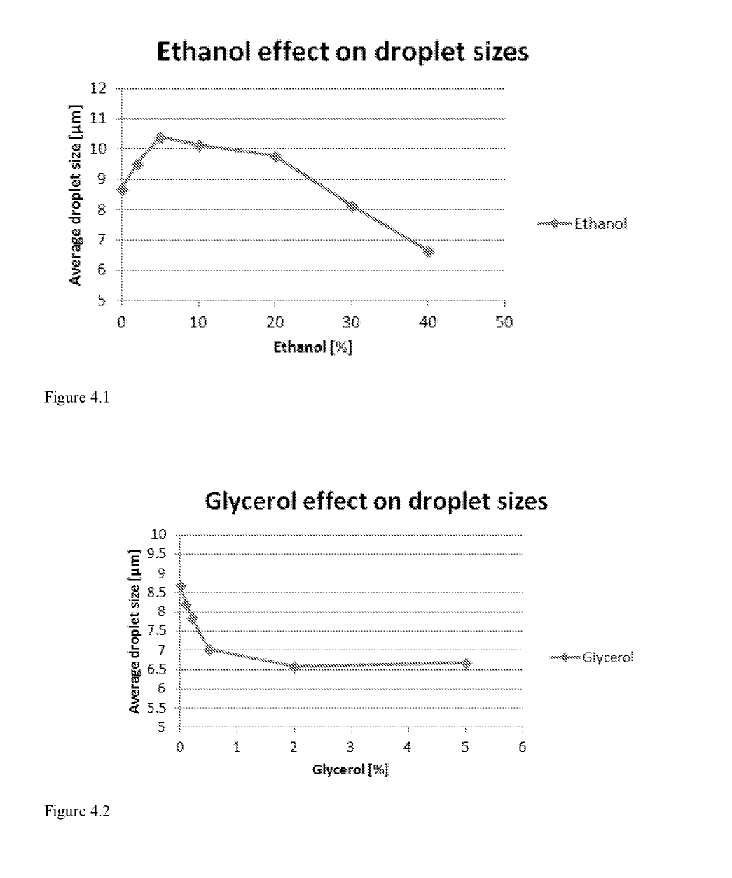

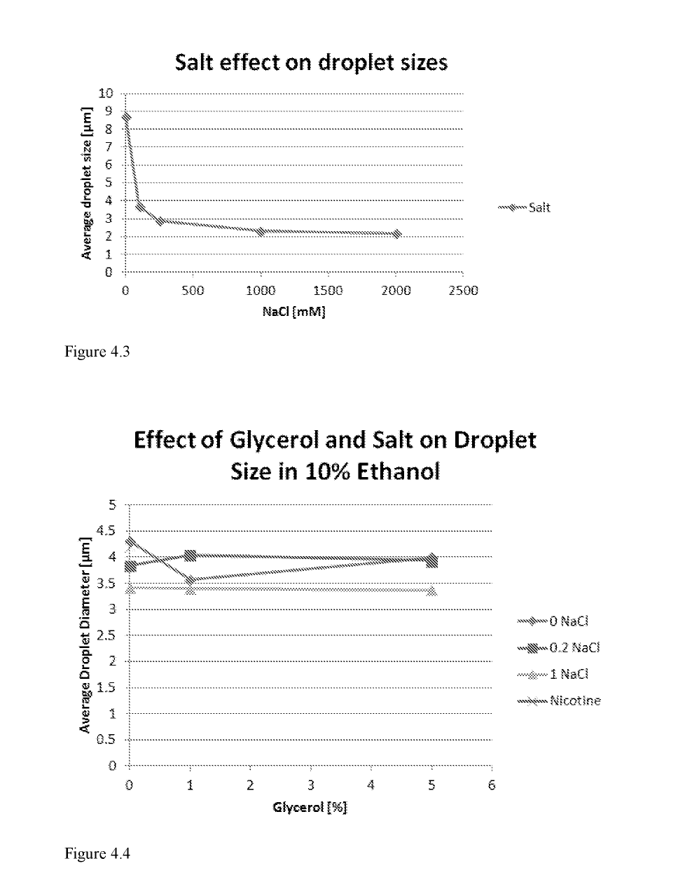

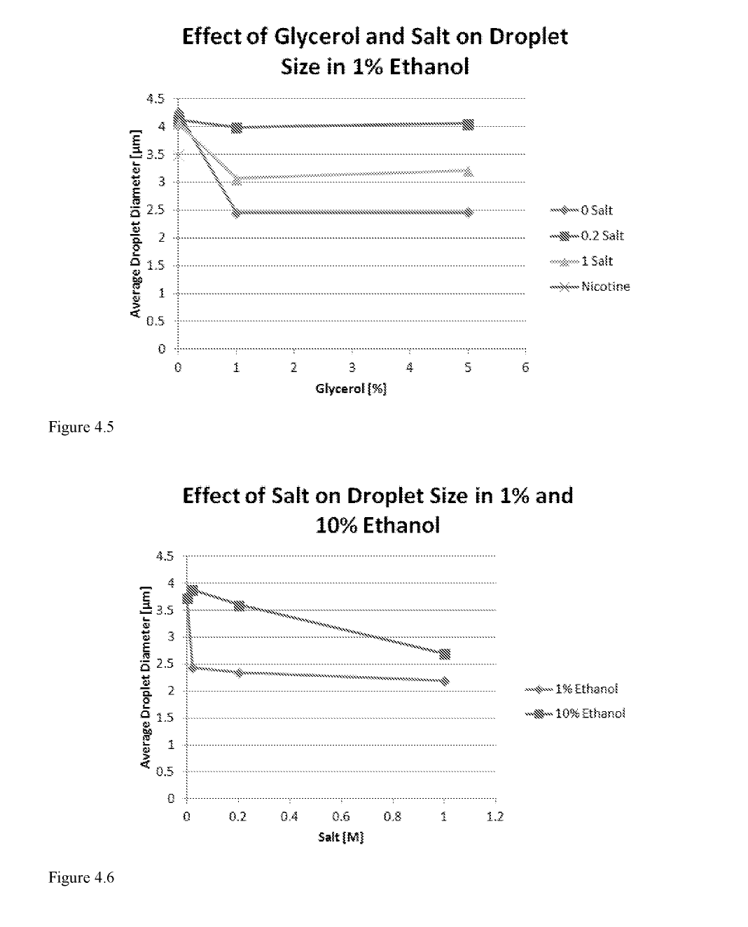

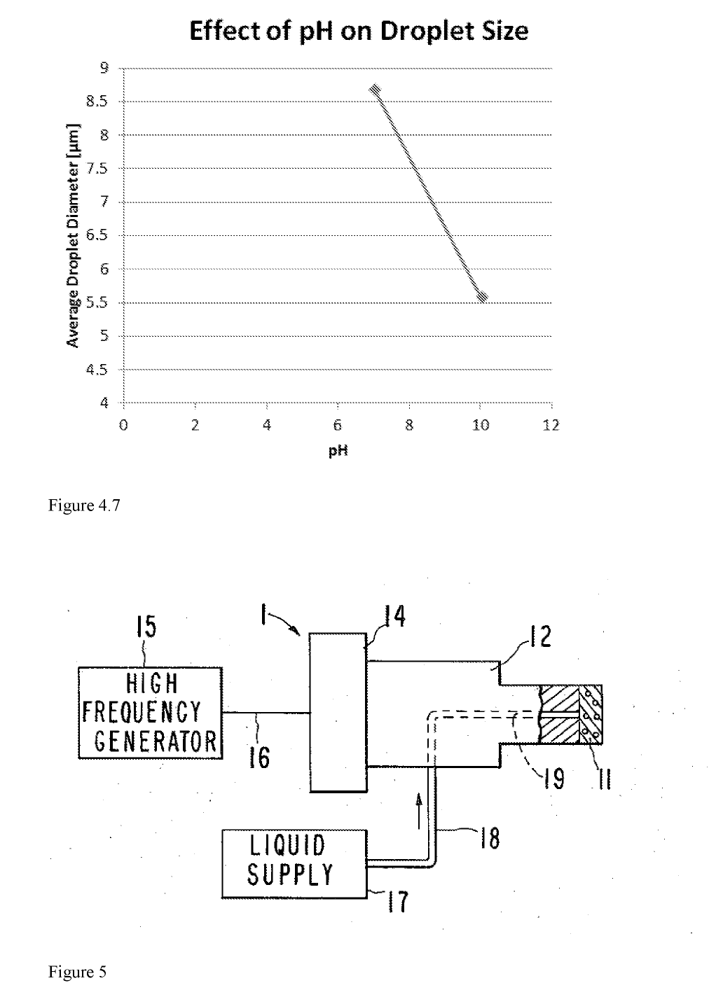

Optimal formulations for nicotine release and droplet targeting comprise of multi-factorial and overlapping influences among the ingredient classes specified. For example, a small variation in ethanol concentration can be off-set by a compensating alteration in glycerol content and vice versa. FIG. 4.1 demonstrates that increasing ethanol from 0% to 10% significantly increases the average droplet sizes generated, with most of the observed increase at low concentrations. While FIG. 4.2 demonstrates that increasing glycerol from 0% to 5% significantly decreases the average droplet sizes generated, with most of the size reduction effected at 1-2% glycerol. FIG. 4.3 demonstrates that increasing NaCl concentration from 0 to 2M significantly decreases the average droplet sizes generated, with 80% of reduction occurring with just 200 mM NaCl. FIG. 4.4 demonstrates that increasing NaCl concentration from 0.02 to 1M decreases the average droplet sizes generated by 10%, with glycerol having little effect. The droplet generation including 20 mg/ml nicotine in the formulation: no major shift in droplet size by addition of the maximum amount of nicotine indicates droplet sizing is applicable to predicting nicotine droplet generation. However, FIGS. 4.5 and 4.6 demonstrate that increasing glycerol concentration from 0 to 5% significantly decreases the average droplet sizes generates, with 80% of reduction occurring with just 1-2% glycerol. The droplet generation including 20 mg/ml nicotine in the formulation: no major shift in droplet size by addition of maximum amount of nicotine indicates droplet sizing applicable to predicting nicotine droplet generation. FIG. 4.7 demonstrates that increasing pH from 7 (unbuffered) to 10 dramatically reduced droplet size. At 0.4 mM NaOH, a significant contribution of 400 nanomolar Na⁺ ions is, while possible, not likely.

In addition, the inventors have found that increasing salt content causes droplet size increase at high humidity, while solvents with a lower polarity than water will slow release of dissolved uncharged gases like nicotine. Furthermore, that a highly effective combination of generating small droplets using glycerol/ethanol mixtures to target alveoli (with the most efficient gas exchange mechanism) with the salt and pH driven high nicotine gas release and the EEG driven prolonged resident-time in the alveoli will deliver the maximum amount of nicotine quickly.

The inventors have also found that by decreasing the density and surface tension of an aqueous solution e.g. by the addition of ethanol, droplet size will increase when generated as aerosol sprays from fluids forced under pressure through one or more nozzles and allowed to impact an external baffle, or 2 or more such streams impacting upon one another.

The system employed here may use a chamberless, planar nozzle plate driven by a piezoelectric actuator, and the inertial transfer mechanism generates a highly defined aerosol of liquid droplets at the touch of a button.

Surface to Volume Ratios

The inventors have found that the effect of gaseous nicotine release is very pronounced if the surface to volume ratio of the droplets generated is high, i.e. the nicotine release is strongest with droplets having an average diameter of less than 10 micrometers, in particular 5 micrometers. Accordingly, effective nicotine delivery may be achieved by using a high pH, a lower nicotine concentration and large surface to volume droplet populations (i.e. small droplets).

To achieve cigarette-like delivery of nicotine to the brain using an aerosol of an aqueous nicotine solution, arterial blood would need to contain nicotine concentrations similar to those seen in smokers and in a similar time span (of 2-6 minutes). Typically, effective nicotine concentrations in arterial blood peak after 3-5 minutes and reach between 20 and 60 ng/ml nicotine. The human lung typically passes the entire volume of 5 litres of blood through its arteries for gas exchange per minute. Over an average time of 3 minutes to reach peak nicotine levels in arterial blood, i.e. 5000 ml containing 60 ng/ml nicotine, 100 μg of gaseous nicotine have to be absorbed per minute. (100 μg*3/5,000 ml=0.06 μg/ml). According to references Calafat et al., CH, Tob Control 2004; 13:45-51 and Pankow et al., (1997) Environmental Science and Technology 31(8):2428-2433, it is reasonable to assume that most of the gaseous nicotine in the lung would be carried by arterial blood (25% of the average 1 mg of nicotine delivered by a cigarette giving rise to about 30-60 ng/ml nicotine in 5 litres of arterial blood). A formulation of nicotine inhaled as an aerosol would therefore need to allow nicotine gas formation at at least 100 μg per minute. Given an average ‘puff’ volume of 30 ml and assuming a rate of 10 ‘puffs’ per minute, then each puff should contain 10 μg of nicotine in uncharged, gas-capable form. Typically, 2 μl of formulation are aerosolized per puff, resulting in an un-protonated nicotine concentration of approximately 5 mg/ml. While many consumers might appreciate a lower peak concentration than 60 ng/ml nicotine in arterial blood, inefficiencies in gas release and diffusion might result in significantly lower peak concentrations.

The inventors have found that lower concentrations of un-protonated nicotine at high pH or lower pH with higher concentrations of nicotine could be used in the formulation in order to lower the amount of un-protonated nicotine administered. The inventors have also found that in order for nicotine to move from an aqueous solution into the gas phase the nicotine had to be un-protonated and in addition, the area from which the nicotine could enter the gas phase had to be large in relation to the volume in which it was dissolved. When a formulation of 2 mg/ml nicotine in 100 mM NaCl, 1% glycerol at pH 9.8 was aerosolized using a PART eFlow device and analysed for droplet size using an Alberta Idealized Throat/Andersen Cascade Impactor at standard (30 l/min) air flow rates, larger droplets deposited at earlier stages contained a larger proportion of the originally present nicotine compared to the smaller ones collected at later stages. At the air flow rates used, the droplets collected at stages 0 and 1 correspond to droplets of a diameter of about 10 μm to 6 μm and the droplets collected at stages 4-6 correspond to droplet diameters of 3.5 μm to <1 μm. The generated droplets had a mean diameter of 3.3 μm (S/V ratio of 1.82 μm⁻¹).

By contrast, droplets generated, collected and analysed in the same fashion but from formulation which had a pH of 3.0, exhibited an almost identical droplet distribution, but with most of the nicotine still present in the collected droplets at each stage. Droplet size distributions were verified using a Malvern Spraytec Particle Sizer. As such, the formulation at pH 9.8 was found to release >95% more of its nicotine in a simulated lung inhalation model (AIT/ACl) than the aerosol with a similar droplet size distribution, but a pH of 3.0. The nicotine release was measured from a distinct fraction of droplet sizes generated by a nebuliser and fractionated by an Alberta Idealised Throat coupled with an Andersen Cascade Impactor, both operated at 30 l/min air flow.

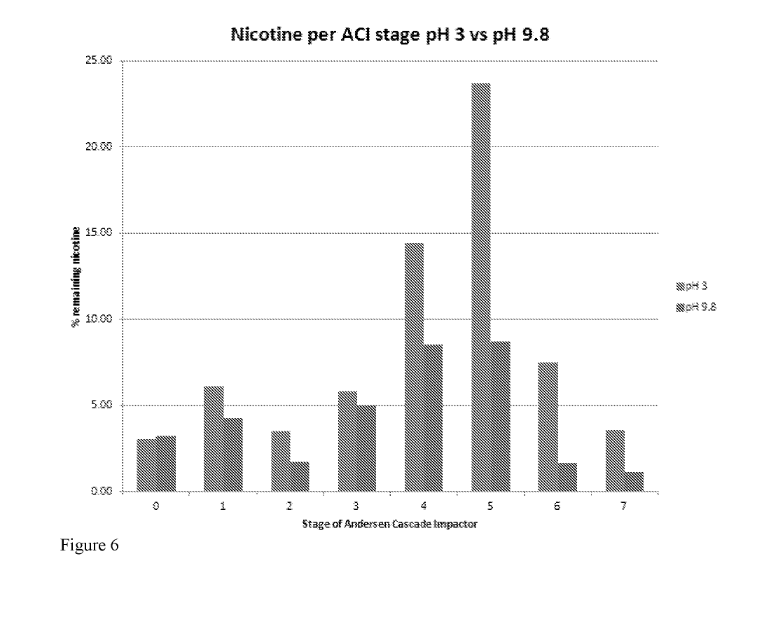

FIG. 6 depicts the results of an experiment comparing the nicotine remaining in the droplets collected at various stages in an Andersen Cascade Impactor when formulations with different pH are used to generate an aerosol using a PARI eFlow device and a flow rate of 30 l/min for 30 seconds. (The formulation contains nicotine, NaCl, glycerol and ethanol in the amounts specified above) While the values for the formulation with a pH of 3 follow the droplet size distribution, the values for the droplets from the formulation with a pH of 9.8 do not, with significant amounts of nicotine missing, especially from stages 4-7, i.e. from droplets with a diameter smaller than 3.5 μm.

The present invention therefore teaches a novel aqueous formulation of nicotine which can deliver 40 to 100 μs per minute of uncharged nicotine in gas form to the lungs when inhaled in the form of droplets with a surface to volume ratio of greater than 0.6 μm⁻¹. As such, cigarette-like nicotine pharmacokinetics and resulting user sensations can be achieved in about 3 minutes using substantially less nicotine than used in current formulations.

1.-31. (canceled)

32. A liquid formulation for inhalation into lungs, the liquid formulation comprising:
an aqueous solution of nicotine or a nicotine analogous molecule capable of binding to a Nicotinic Acetylcholine Receptor, an organic salt or an inorganic salt, an organic liquid having a viscosity higher than water, and less than 50% vol/vol of an organic alcohol,
wherein a pH of the liquid formulation is greater than pH 7 and the formulation does not include a propellant.

33. The liquid formulation of claim 32, wherein the pH of the liquid formulation is between pH 7.5 and 14.

34. The liquid formulation of claim 32, wherein the liquid formulation contains between 0 and 20 μg/μl of the nicotine or the nicotine analogous molecule.

35. The liquid formulation of claim 32, wherein the pH of the liquid formulation is between pH 7.5 and 8.5 and the liquid formulation contains between 8 μg/μl and 20 μg/μl of the nicotine or the nicotine analogous molecule.

36. The liquid formulation of claim 32, wherein the pH of the liquid formulation is between pH 8.5 and 14 and the liquid formulation contains nicotine in an amount of between 0 μg/μl and 8 μg/μl of the nicotine or the nicotine analogous molecule.

37. The liquid formulation of claim 32, wherein the organic salt or the inorganic salt is selected from a group consisting of sodium chloride, metal citrates, metal sulphates and combinations thereof.

38. The liquid formulation of claim 32, wherein the organic liquid having a viscosity higher than water is selected from a group consisting of glycerol, glycol, a carbohydrate solution and combinations thereof.

39. The liquid formulation of claim 32, wherein the organic alcohol is ethanol or a diol.

40. The liquid formulation of claim 32, wherein the liquid formulation does not contain ethanol.

41. The liquid formulation of claim 32, wherein the organic liquid having a viscosity higher than water is included in a range of 0.5 to 10% vol/vol.

42. The liquid formulation of claim 32, wherein a combined concentration of the organic salt or the inorganic salt is between 0.05 molar and 2 molar.

43. The liquid formulation of claim 32, wherein the organic alcohol is ethanol, and wherein the liquid formulation contains less than 50% of the ethanol.

44. The liquid formulation of claim 32, wherein the liquid formulation comprises:
between 2 mg/ml and 20 mg/ml of the nicotine or the nicotine analogous molecule;
between 0.05M and 2M concentration of the organic salt or the inorganic salt;
between 0.5% vol/vol and 2% vol/vol of the organic liquid having a viscosity higher than water, wherein the organic liquid is at least one of glycerol and glycol; and
between 1% vol/vol and 10% vol/vol of the organic alcohol, wherein the organic alcohol is ethanol.

45. The liquid formulation of claim 32, further comprising an additive, wherein the additive is at least one of a flavouring, a colourant, an antioxidant, and a surfactant.

46. The liquid formulation of claim 32, wherein the liquid formulation forms an aerosol when dispensed with an aerosol-generating device.

47. The liquid formulation of claim 46, wherein the aerosol contains droplets of the liquid formulation, wherein the droplets have a mean diameter between 10 μm and 0.4 μm or a surface to volume ratio between 0.6 μm⁻¹ and 15 μm⁻¹.

48. The liquid formulation according to claim 47, wherein the liquid formulation comprises between 1.5 μg/μl and 8 μg/μl of the nicotine or the nicotine analogous molecule.

49. An aerosol-generating device comprising a reservoir which contains the liquid formulation of claim 32.

50. The aerosol-generating device of claim 49, wherein the device is configured to generate an average droplet size of between 5 μm and 400 nm, when activated to aerosolise the liquid formulation.

51. The aerosol-generating device of claim 49, wherein the device is configured to generate a droplet size having an average surface to volume ratio of between 0.6 μm⁻¹ and 30 μm⁻¹ when activated to aerosolise the liquid formulation.

52. A method of forming an aerosol, comprising the steps of:
providing the liquid formulation of claim 32; and
aerosolising the liquid formulation.

53. The method of forming an aerosol of claim 52, wherein the aerosol is administered to a user and the nicotine or the nicotine analogous molecule is delivered to arterial blood of the user.
